SEQUENCE LISTING (22 sequences)

SEQ ID NO: 1 - DNA, 5380 bp, Artificial Sequence
  Description of Artificial Sequence Synthetic construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattcgattatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaatcgtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 2 - DNA, 4883 bp, Artificial Sequence
  Description of Artificial Sequence Synthetic construct
gcaacaactccgccccattgacgcaaatgggcggtaggcgtgtacggtgggaggtctatataagcagagctggtttagtgaaccgtcagatccgctagcgctaccggactcagatctcgagctcaagcttcgaattcgattatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaatcgtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 3 - DNA, 23 bp, Artificial Sequence
  Description of Artificial Sequence Synthetic primer
ctgattttacggaggttgatgtc

SEQ ID NO: 4 - DNA, 24 bp, Artificial Sequence
  Description of Artificial Sequence Synthetic primer
tcctttctctttagcagacatggt

SEQ ID NO: 5 - DNA, 25 bp, Artificial Sequence
  Description of Artificial Sequence Synthetic primer
aggtaagagggcttaaaacatgcta

SEQ ID NO: 6 - DNA, 25 bp, Artificial Sequence
  Description of Artificial Sequence Synthetic primer
cccacccaacaggaattttatacta

SEQ ID NO: 7 - DNA, 25 bp, Artificial Sequence
  Description of Artificial Sequence Synthetic primer
tatagtatttgcaccctgtccaatg

SEQ ID NO: 8 - DNA, 24 bp, Artificial Sequence
  Description of Artificial Sequence Synthetic primer
accctaatttatttggtcctctgc

SEQ ID NO: 9 - DNA, 26 bp, Artificial Sequence
  Description of Artificial Sequence Synthetic primer
ggatctacagatacgtgatattttgg

SEQ ID NO: 10 - DNA, 25 bp, Artificial Sequence
  Description of Artificial Sequence Synthetic primer
gacagggctatctaaagacacattc

SEQ ID NO: 11 - DNA, 18 bp, Artificial Sequence
  Description of Artificial Sequence Synthetic primer
atgggcaaaggagatcct

SEQ ID NO: 12 - DNA, 30 bp, Artificial Sequence
  Description of Artificial Sequence Synthetic primer
agcagacatggtcttccacctctctgagca

SEQ ID NO: 13 - DNA, 22 bp, Artificial Sequence
  Description of Artificial Sequence Synthetic primer
ggaagaccatgtctgctaaaga

SEQ ID NO: 14 - DNA, 17 bp, Artificial Sequence
  Description of Artificial Sequence Synthetic primer
cttgggtgcattgggat

SEQ ID NO: 15 - DNA, 38 bp, Artificial Sequence
  Description of Artificial Sequence Synthetic primer
cccaatgcacccaagaggcctccttcggccttcttcct

SEQ ID NO: 16 - DNA, 31 bp, Artificial Sequence
  Description of Artificial Sequence Synthetic primer
gcagcaatatccttttcgtatttttccttca

SEQ ID NO: 17 - DNA, 26 bp, Artificial Sequence
  Description of Artificial Sequence Synthetic primer
acgaaaaggatattgctgcatatcga

SEQ ID NO: 18 - DNA, 18 bp, Artificial Sequence
  Description of Artificial Sequence Synthetic primer
tgcgctagaaccaactta

SEQ ID NO: 19 - protein, 6 aa, Artificial Sequence
  Description of Artificial Sequence Synthetic peptide
KKKRKV

SEQ ID NO: 20 - protein, 16 aa, Artificial Sequence
  Description of Artificial Sequence Synthetic peptide
KRPAATKKAGQAKKKK

SEQ ID NO: 21 - protein, 16 aa, Artificial Sequence
  Description of Artificial Sequence Synthetic peptide
RQIKIWFQNRRMKWKK

SEQ ID NO: 22 - protein, 13 aa, Artificial Sequence
  Description of Artificial Sequence Synthetic peptide
GRKKRRQRRRPPQ

(19) United States
(12) Patent Application Publication
(10) Pub. No.: US 2010/0055701 A1
(43) Pub. Date: Mar. 4, 2010

(54) FUSION PROTEINS FOR IMAGING THE NUCLEUS AND CHROMOSOMES OF LIVE CELLS

(76) Inventors: YIGANG TONG, BEIJING (CN); XIAOPING AN, BEIJING (CN); XIN ZHANG, BEIJING (CN)

Correspondence Address: FOLEY & LARDNER LLP, 150 EAST GILMAN STREET, P.O. BOX 1497, MADISON, WI, 53701-1497, US

(21) Appl. No.: 12/542,290

(22) Filed: Aug. 17, 2009

(30) Foreign Application Priority Data
Aug. 27, 2008 (CN) 200810210548.3

(51) Int. Cl. C12Q 1/68; C07K 1/00; C12N 5/00; C12N 15/63

(52) U.S. Cl. 435/6; 530/402; 435/325; 435/320.1

(57) ABSTRACT

Fusion proteins that fluoresce and bind DNA, as well as nucleic acid constructs encoding the same are described herein. These imaging agents function in live cells to bind DNA and are thus useful for visualizing nuclei and chromosomes to make observations of nuclear and chromosomal structure and dynamics. The imaging agents may be used in screening assays to test the activity of biological effector molecules.

### CROSS-REFERENCE TO RELATED APPLICATIONS

This application claims priority to Chinese Patent Application No. 200810210548.3, filed Aug. 27, 2008, the entire contents of which are hereby incorporated by reference in its entirety.

### BACKGROUND

The present technology relates generally to the fields of cell biology, molecular biology, and pharmacology.

### SUMMARY

One aspect of the present technology includes an imaging agent comprising a fusion protein having one or more DNA binding domains and one or more fluorescent domains, wherein the fusion protein is configured to localize in the nucleus of a cell. In some embodiments, the cell is a live cell. In some embodiments, the DNA binding domain is selected from the group consisting of: an HMGB1; an HMGB2; and a histone H1. In some embodiments, the fluorescent domain is selected from the group consisting of: a GFP; an EGFP; a YFP; an EYFP; a CFP; an ECFP; a BFP; an EBFP; an RFP; and a DsRed. In an illustrative embodiment, the DNA binding domain is HMGB1 and the fluorescent domain is EGFP.

In some embodiments, the fusion protein further comprises a nuclear localization domain. In an illustrative embodiment, the nuclear localization domain is a nuclear localization sequence from the SV40 virus. In some embodiments, the fusion protein further comprises a linker peptide between the DNA binding domain and the fluorescent domain. In some embodiments, the fusion protein further comprises a cell-penetrating peptide. In illustrative embodiments, the cell-penetrating peptide is selected from the group consisting of: an antennapedia; a TAT; a transportan; and a polyarginine. Another aspect of the present technology includes a cell comprising the imaging agent.

Another aspect of the present technology includes a nucleic acid construct comprising a promoter operably linked to one or more DNA binding domains, one or more fluorescent domains and at least one nuclear localization domain. In some embodiments, the promoter is selected from the group consisting of: a strong constitutive promoter; a weak constitutive promoter; a tissue-specific promoter; and an inducible promoter. In an illustrative embodiment, the strong constitutive promoter is a CMV early promoter. In an illustrative embodiment, the weak constitutive promoter is a truncated CMV early promoter.

Another aspect of the present technology includes vectors comprising a nucleic acid construct comprising a promoter operably linked to one or more DNA binding domains, one or more fluorescent domains and at least one nuclear localization domain. In an illustrative embodiment, the vector has the sequence according to SEQ ID NO: 1 or SEQ ID NO: 2. This disclosure also pertains to cells transformed or transfected with a nucleic acid vector comprising a promoter operably linked to one or more DNA binding domains, one or more fluorescent domains and at least one nuclear localization domain.

Another aspect of the technology includes a method for imaging comprising: (a) introducing into a cell: (i) an imaging agent of the present technology or (ii) a nucleic acid construct, wherein the construct is capable of expressing an imaging agent of the present technology in the cell; and (b) detecting the fluorescence of the imaging agent. In some embodiments, the step of detecting comprises imaging the nucleus of a cell. In some embodiments, the step of detecting comprises imaging the chromosomes of a cell.

Another aspect of the technology includes a method for monitoring one or more nucleus-associated changes in a cell comprising: (a) introducing into a test cell: (i) an imaging agent of the present technology or (ii) a nucleic acid construct encoding the imaging agent, wherein the construct is capable of expressing an imaging agent of the present technology in the cell; and (b) detecting one or more changes in fluorescence of the imaging agent in the test cell, wherein one or more changes in fluorescence between the fluorescence of the imaging agent in the test cell compared to a reference fluorescence is indicative of one or more nucleus associated changes in the cell. In some embodiments, the one or more nucleus associated changes include one or more chromosomal aberrations. In some embodiments, the one or more chromosomal aberrations are selected from: deletion, duplication, inversion, and translocation. In some embodiments, the one or more nucleus associated changes are indicative of cell apoptosis or cell division. In illustrative embodiments, the one or more changes in fluorescence include one or more changes in the level or distribution of fluorescence.

Another aspect of the technology includes a method for observing one or more effects of one or more molecules on a cell comprising: (a) introducing into a test cell: (i) the imaging agent of the present technology or (ii) the nucleic acid construct, wherein the construct is capable of expressing an imaging agent of the present technology in the cell; (b) contacting the test cell with one or more molecules; and (c) detecting one or more changes in fluorescence of the imaging agent in the test cell, wherein one or more changes in fluorescence between the fluorescence of the imaging agent in the test cell compared to a reference fluorescence is indicative of one or more effects of the one or more molecules on the test cell.

In some embodiments, the effect of the one or more molecules being observed is toxicity leading to death of the cell or chromosomal abnormality. In some embodiments, the one or more molecules include one or more of the molecules selected from the group consisting of: therapeutic agents; pesticides; herbicides; compounds; small molecules toxins; nucleic acid; proteins; and peptides.

Another aspect of the technology includes kits for imaging comprising one or more of the imaging agent, the nucleic acid construct encoding the imaging agent of the present technology, vectors, and/or cells as described herein.

### BRIEF DESCRIPTION OF THE DRAWINGS

FIG. 1 is a map of the HMGB1-EGFP fusion protein construct in the pEGFP-lac vector.

FIG. 2 shows photomicrographs demonstrating nuclear localization of the HMGB1-EGFP fusion protein in both HEK 293FT and CHO cells.

FIG. 3 shows photomicrographs demonstrating that apoptosis results in round cell shape and diffusion of HMGB1-EGFP throughout the cell.

FIG. 4 is a map of the ptrEGFP-HMGB1 expression plasmid.

FIG. 5A and FIG. 5B show photomicrographs demonstrating that the ptrEGFP-HMGB1 expression plasmid allows for visualization of chromosomes.

### DETAILED DESCRIPTION

Disclosed herein are proteins, nucleic acids, cells, and methods for visualizing the nuclei and chromosomes of live cells. In particular, the present technology provides fusion proteins that fluoresce and bind DNA. These fusion proteins function in live cells to bind DNA; they are thus useful for visualizing nuclei and chromosomes to make observations of nuclear and chromosomal structure and dynamics.

Researchers often need to know the growth status of cells and cell cultures. This status can be determined by observing the shape and size of cells and their nuclei because nuclear structure is an early marker for many cellular events of interest. For instance, marked changes in nuclear and chromosome structure signal the onset of apoptosis, mitosis, meiosis, and cell division. Observing these changes is important for studying and diagnosing human disease states including, but not limited to cancer.

Hoechst dyes are often used to stain cells to make these types of observations. However, these dyes are toxic to cells and are often used on fixed, and therefore, dead cells. This disclosure describes a protein that can display the cell nucleus in live cells without causing harm to the cells. Moreover, the protein can clearly show the shape and position of condensed chromosomes. The ability to visualize condensed chromosomes is particularly relevant, for example, when cells are preparing for cell division or are undergoing apoptosis.

In the description that follows, a number of terms are utilized extensively. The explanations are herein provided to facilitate understanding of the invention. The terms provided below are more fully explicated by reference to the specification as a whole.

Units, prefixes, and symbols may be denoted in their accepted SI form. Unless otherwise indicated, nucleic acids are written left to right in 5′ to 3′ orientation; amino acid sequences are written left to right in amino to carboxy orientation. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes.

The terms “a” and “an” as used herein mean “one or more” unless the singular is expressly specified or context clearly dictates otherwise.

As used herein, a “cell-penetrating peptide” refers to a peptide having the ability to transduce another peptide, protein, or nucleic acid into a cell in vitro and/or in vivo.

As used herein, a “DNA-binding protein” refers to a protein that associates with DNA and that may have a general affinity for DNA or a specific affinity for particular DNA sequences.

As used herein, “expression” refers to the process by which a polypeptide is produced from a structural gene. Overall, the process involves transcription of a gene into RNA and the translation of such RNA into polypeptide(s).

As used herein, a “fluorescent protein” refers to a protein that fluoresces at a characteristic wavelength of emission when exposed to electromagnetic radiation of an appropriate wavelength of excitation.

As used herein, an “imaging agent” refers to any substance used for visually reporting a cell's state or the state of subcellular structures or organelles without otherwise generally affecting the cell.

As used herein, the terms “linked,” “conjugated,” “fused” or “fusion” are used interchangeably. These terms refer to the joining together of two more elements or components, by whatever means including chemical conjugation or recombinant means. A fusion protein refers to a single protein containing two or more segments that correspond to polypeptides which are not normally so joined in nature. The segments may be physically or spatially separated by, for example, a linker peptide sequence.

As used herein, an “inducible promoter” refers to a promoter that is sensitive to the presence of a stimulus (e.g., heat shock, chemicals, etc.). The stimulus directs a level of transcription of an operably linked nucleic acid sequence that is higher or lower than the level of transcription of the operably linked nucleic acid sequence in the absence of the stimulus.

As used herein, a “nuclear localization sequence” (NLS) refers to a polypeptide capable of directing the localization of a protein or polynucleotide to the nucleus of a cell.

As used herein, a “nucleic acid” refers to a deoxyribonucleotide or ribonucleotide polymer, or chimeras thereof, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

As used herein, “operably linked” refers to a functional linkage between two sequences. For example, when a promoter and a structural gene are joined, the promoter sequence initiates and mediates transcription of the structural gene. The term “operably linked” also means that the nucleic acid sequences being linked are contiguous and, where necessary to join two or more protein coding regions (optionally including a linker peptide), the sequences are contiguous and in the same reading frame.

As used herein, the terms “polypeptide,” “peptide,” and “protein” are used interchangeably to refer to a polymer of amino acid residues. The terms apply to amino acid polymers in which one or more amino acid residues is an artificial chemical analog of a corresponding naturally occurring amino acid, as well as to polymers having only naturally occurring amino acid polymers. The terms polypeptide, peptide, and protein are also inclusive of modifications including, but not limited to, e.g., glycosylation, lipid attachment, sulfation, carboxylation, hydroxylation, ADP-ribosylation, and addition of other complex polysaccharides.

As used herein, a “promoter” refers to a DNA sequence that directs the transcription of a structural gene to produce a messenger RNA (mRNA). Typically, a promoter is located in the 5′ region of a gene, proximal to the start codon. If a promoter is an inducible promoter, then the rate of transcription increases in response to an inducing agent compared to the rate of transcription in the absence of the inducing agent. In contrast, the rate of transcription is not regulated by an inducing agent if the promoter is a constitutive promoter.

As used herein, a “strong constitutive promoter” refers to a promoter that is active under most conditions and directs a high rate of transcription of an operably linked nucleic acid sequence. “High rate of transcription” means that from about 1/10 transcripts to about 1/100 transcripts to about 1/1,000 transcripts in the cell will correspond to the operably linked nucleic acid sequence.

As used herein, a “truncated promoter” refers to a promoter that is shortened by removing a portion of its normal sequence, usually to render it to be less active and thus to decrease the amount of protein expressed from the nucleic acid operably linked to the truncated promoter compared to the amount of protein expressed from the corresponding full-length promoter.

As used herein, a “vector” refers to a DNA molecule, such as a plasmid, cosmid, phagemid, or bacteriophage, which has the capability of replicating autonomously in a host cell and which is used to transform or transfect cells for gene manipulation. Expression vectors permit transcription of a nucleic acid inserted therein.

As used herein, a “weak constitutive promoter” refers to a promoter that is active under most conditions and directs a low rate of transcription of an operably linked nucleic acid sequence. “Low rate of transcription” means that from about 1/10,000 transcripts to about 1/100,000 transcripts to about 1/500,000 transcripts in the cell will correspond to the operably linked nucleic acid sequence.

### I. Imaging Agent Compositions

A. Fusion Proteins

In one aspect, this disclosure provides a detectable imaging agent that binds to DNA. In particular, this disclosure provides proteins, nucleic acids, cells, and methods useful for visualizing live cells under physiological conditions. The imaging agent comprises a DNA-binding domain associated with a fluorescent domain. The imaging agent can enter the nucleus of a living cell and bind to DNA.

The imaging agents comprise DNA binding domains. DNA binding domains are proteins that have a specific or general affinity for DNA. In some embodiments, sequence-specific DNA binding proteins are used in the imaging agents. Sequence-specific DNA-binding proteins typically recognize and bind to particular sequences of nucleotides (e.g., the TATA binding protein). In other embodiments, non-sequence-specific DNA binding proteins are used in the imaging agents. Non-sequence-specific DNA-binding proteins have a general affinity for DNA—that is, they typically do not have a substantial preference for any particular DNA sequence, but instead bind with the DNA double helix through gross structural and electrostatic interactions. Non-sequence-specific DNA-binding proteins will thus bind to any DNA molecule.

In some embodiments, the DNA-binding domain of the protein imaging agent is a non-sequence-specific DNA-binding protein. Some non-sequence-specific DNA-binding proteins include the human high-mobility group protein B1 (HMGB1), human high-mobility group protein B2 (HMGB2), other HMG1 proteins, other HMG2 proteins, histone H1, Sso7d, and other homologues of these proteins. HMGB1 and HMGB2 are normally expressed in the cell nucleus. HMGB1 is present in all vertebrate nuclei and is very highly conserved; for instance, mouse and rat HMGB1 are identical and differ from human HMGB1 at only two positions (Andersson et al., J Leukoc Biol. December 2002;72(6): 1084-91). Thus, any particular variant of HMGB1 is expected to be functional in most cell types. In other embodiments, the DNA-binding protein may be a histone. Histones, the main protein components of chromatin, are present in the nuclei of cells and are highly conserved. In one embodiment, the histone is histone H1.

The imaging agents include a fluorescent protein domain to allow for visualization of the nucleus and chromosomes in a live cell. Fluorescent proteins have particular arrangements of amino acids that give the protein its fluorescent properties. By virtue of these amino acids, when the protein is excited by photons of a particular wavelength, the protein emits photons of a different, longer wavelength. Typically, fluorescent proteins are excited by photons in the ultraviolet region and emit photons in the visible region. In some embodiments, the fluorescent protein domain is a green fluorescent protein (GFP). The maxima of the excitation and emission spectra for GFP are approximately 495 nm and 509 nm, respectively. Thus, GFP is a protein that fluoresces green when exposed to blue light. GFP folds and fluoresces at room temperature without the need for other cofactors or reagents, and functions in a wide variety of organisms and cell types including, but not limited to, bacteria, yeast, fungi, plant, insects, worms, mammals, and humans.

Variants of GFP and other fluorescent proteins with characteristic excitation and emission spectra are also useful. In some embodiments, the fluorescent protein may be a yellow fluorescent protein (YFP), a red fluorescent protein (RFP), a Discosoma sp. red fluorescent protein (DsRed), a cyan fluorescent protein (CFP), a blue fluorescent protein (BFP), an enhanced green fluorescent protein (EGFP), an enhanced yellow fluorescent protein (EYFP), an enhanced cyan fluorescent protein (ECFP), or an enhanced blue fluorescent protein (EBFP). In yet other embodiments, the fluorescent protein is an optimized mutant of a fluorescent protein. Examples of other fluorescent proteins are the S65T GFP mutant, Superfolder GFP, Azurite, mKalama1, Cerulean, CyPet, Citrine, Venus, and YPet.

In an illustrative embodiment, the fusion protein of HMGB1 and EGFP is an imaging agent for visualizing live cells.

The fluorescent protein domain and DNA-binding domain may be associated with one another by being present on the same polypeptide. In this embodiment, the DNA-binding protein and fluorescent protein domain are fused in the same reading frame such that the coding sequence of one domain immediately or closely follows the coding sequence of the other.

In some embodiments, the fluorescent domain and the DNA-binding domain may be associated with one another by a linker joining the two domains. The linker may be a peptide or suitable chemical group. The linker may have no function except to connect physically two proteins or polypeptides. In one embodiment, the linker is a peptide that is translated in frame with the fluorescent domain and the DNA binding domain. The linker moiety should be long enough and flexible enough to allow the DNA binding domain to bind DNA without steric hindrance from the fluorescent domain. The linker moiety is optionally a peptide moiety. The linker moiety is optionally a peptide between about one and 30 amino acid residues in length, optionally between about two and 15 amino acid residues. In an illustrative embodiment, the linker moiety is a -Gly--Gly-linker. Linking moieties are described, for example, in Huston et al., PNAS 85:5879-5883, 1988, Whitlow et al., Protein Engineering 6:989-995, 1993, and Newton et al., Biochemistry 35:545-553, 1996.

In some embodiments the fluorescent domain and DNA-binding domain may be separate polypeptides that self-associate in vitro or in vivo. Self-association may be mediated by any useful protein dimerization domain or coupling agent known to one skilled in the art. An example of a protein dimerization domain is a leucine zipper, which mediates the association of the AP1 transcription factor. Examples of coupling agents that may be used to mediate protein self-association are avidin-biotin, antibody-antigen pairs, and receptor-ligand pairs. The avidin-biotin complex is known to those of skill in the art as one of the strongest non-covalent associations between two molecules. To use avidin-biotin for the self-association of the imaging agent in vitro or in vivo, avidin may be linked to either the DNA-binding or fluorescent domain and biotin may be linked to the other domain. After introduction of the modified domains into a cell, the domains self-associate by virtue of the avidin-biotin complex, thus constituting the imaging agent. A chemical linker may also include any chemical group that can join two polypeptides, such as an amide, an ester, a thioester, a phosphoester, a phosphoramide, an anhydride, a disulfide, cross-linking agents, and other linkages known to those skilled in the art.

The imaging agents can also include a nuclear localization domain to direct the imaging agent to the nucleus of the cell by fusion to appropriate organellar targeting signals or localized host proteins. A polynucleotide encoding a localization sequence, or signal sequence, can be ligated or fused at the 5′ terminus of a polynucleotide encoding the imaging agent such that the signal peptide is located at the amino terminal end of the resulting fusion polynucleotide/polypeptide.

For example, the imaging agent of any of these embodiments may include a nuclear localization sequence (NLS). An NLS causes the protein to which it is attached to be imported into the cell nucleus. Therefore, when the imaging agent includes an NLS, the imaging agent primarily localizes to the nucleus without significantly leaking into the cytoplasm. An NLS consists of one or more short sequences of positively charged lysines or arginines, for example KKKRKV (SEQ ID NO: 19) or KRPAATKKAGQAKKKK (SEQ ID NO: 20). These signals are bound by importins, which import the NLS-containing protein through the nuclear pore and into the cell nucleus. Nuclear localization sequences may be derived, for example, from an SV40 large T antigen, a nucleoplasmin, a Chelsky sequence, a C-myc, an M9 domain of hnRNP A1, a yeast transcription repressor Matα2, and from a complex signal of a U snRNP.

The imaging agent of any of these embodiments may also include a cell-penetrating peptide. Cell-penetrating peptides facilitate the movement of proteins to which they are attached across the plasma membrane and into cells. Cell-penetrating peptides may include a short polycationic sequence, for example RQIKIWFQNRRMKWKK (SEQ ID NO: 21) or GRKKRRQRRRPPQ (SEQ ID NO: 22). Illustrative examples of cell-penetrating peptides include an antennapedia, a penetratin, a TAT, a transportan, a Pep-1, an S4₁₃-PV, and a polyarginine.

B. Nucleic Acids

In another aspect, this disclosure provides a nucleic acid construct that encodes an imaging agent that is capable of binding DNA and that can be expressed in a live cell. The imaging agents can be produced as fusion proteins by recombinant DNA technology.

Recombinant production of proteins involves expressing nucleic acids having sequences that encode the proteins. Nucleic acids encoding imaging agents can be obtained by methods known in the art. For example, a nucleic acid encoding the protein can be isolated by polymerase chain reaction using primers based on the DNA sequence of interest. PCR methods are described in, for example, U.S. Pat. No. 4,683,195; Mullis et al., Cold Spring Harbor Symp. Quant. Biol. 51:263, 1987, and Erlich, ed., PCR Technology, (Stockton Press, NY, 1989). Mutant versions of fluorescent proteins can be made by site-specific mutagenesis of other nucleic acids encoding fluorescent proteins, or by random mutagenesis caused by increasing the error rate of PCR of the original polynucleotide with 0.1 mM MgCl₂ and unbalanced nucleotide concentrations. See, e.g., U.S. patent application Ser. No. 08/337,915, filed Nov. 10, 1994, or International Application PCT/US95/14692, filed Nov. 10, 1995.

The construction of expression vectors and the expression of genes in transfected cells involves the use of molecular cloning techniques also well known in the art. See Sambrook et al., Molecular Cloning—A Laboratory Manual, Cold Spring Harbor Laboratory, Cold Spring Harbor, N.Y., (1989) and Current Protocols in Molecular Biology, F. M. Ausubel et al., eds.

Nucleic acids used to transfect cells with sequences coding for expression of the polypeptide of interest are optionally in the form of an expression vector including expression control sequences operatively linked to a nucleotide sequence coding for expression of the polypeptide. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i. e., ATG) in front of a protein-encoding gene, splicing signals for introns, maintenance of the correct reading frame of that gene to permit proper translation of the mRNA, and stop codons.

Methods which are well known to those skilled in the art can be used to construct expression vectors containing the imaging agent coding sequence and appropriate transcriptional/translational control signals. These methods include in vitro recombinant DNA techniques, synthetic techniques and in vivo recombination/genetic recombination. (See, for example, the techniques described in Maniatis et al., Molecular Cloning A Laboratory Manual, Cold Spring Harbor Laboratory, N.Y., 1989).

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection of DNA as DNA-liposome complexes or calcium phosphate co-precipitates, conventional mechanical procedures such as microinjection, electroporation, insertion of a plasmid encased in liposomes, or viral vectors may be used. Eukaryotic cells can also be cotransfected with DNA sequences encoding the imaging agent, and a second foreign DNA molecule encoding a selectable phenotype, such as the puromycine, neomycin, hygromycin selectable markers, and the the herpes simplex thymidine kinase gene. Another method is to use a eukaryotic viral vector, such as simian virus 40 (SV40) or bovine papilloma virus, to transiently or stably infect or transform eukaryotic cells and express the protein. (Eukayotic Viral Vectors, Cold Spring Harbor Laboratory, Gluzman ed., 1982).

Techniques for the isolation and purification of either microbially or eukaryotically expressed polypeptides may be by any conventional means such as, for example, preparative chromatographic separations and immunological separations such as those involving the use of monoclonal or polyclonal antibodies or antigen.

A variety of host-expression vector systems may be utilized to express an imaging agent coding sequence. These include, but are not limited, to microorganisms such as bacteria transformed with recombinant bacteriophage DNA, plasmid DNA or cosmid DNA expression vectors. Depending on the host/vector system utilized, any of a number of suitable transcription and translation elements, including constitutive and inducible promoters, transcription enhancer elements, transcription terminators, etc. may be used in the expression vector (See, e.g., Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5 K promoter) may be used. Promoters produced by recombinant DNA or synthetic techniques may also be used to provide for transcription of the inserted fluorescent indicator coding sequence.

In bacterial systems, a number of expression vectors may be advantageously selected depending upon the use intended for the fluorescent indicator expressed. For example, when large quantities of the fluorescent indicator are to be produced, vectors which direct the expression of high levels of fusion protein products that are readily purified may be used.

In yeast, a number of vectors containing constitutive or inducible promoters may be used. For a review see, Current Protocols in Molecular Biology, Vol. 2, Ed. Ausubel et al., Greene Publish. Assoc. & Wiley Interscience, Ch. 13, 1988; Grant et al., Expression and Secretion Vectors for Yeast, in Methods in Enzymology, Eds. Wu & Grossman, 31987, Acad. Press, N.Y., Vol. 153, pp. 516-544, 1987; Glover, DNA Cloning, Vol. II, IRL Press, Wash., D.C., Ch. 3, 1986; and Bitter, Heterologous Gene Expression in Yeast, Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and II, 1982. A constitutive yeast promoter such as ADH or LEU2 or an inducible promoter such as GAL may be used (Cloning in Yeast, Ch. 3, R. Rothstein In: DNA Cloning Vol. 11, A Practical Approach, Ed. DM Glover, IRL Press, Wash., D.C., 1986). Alternatively, vectors may be used which promote integration of foreign DNA sequences into the yeast chromosome.

An alternative expression system which could be used to express an imaging agent is an insect system. In one such system, Autographa californica nuclear polyhedrosis virus (AcNPV) is used as a vector to express foreign genes. The virus grows in Spodoptera frugiperda cells. The fluorescent indicator coding sequence may be cloned into non-essential regions (for example, the polyhedrin gene) of the virus and placed under control of an AcNPV promoter (for example the polyhedrin promoter). Successful insertion of the fluorescent indicator coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed, see Smith et al., J. Viol. 46:584, 1983; Smith, U.S. Pat. No. 4,215,051.

Mammalian cell systems which utilize recombinant viruses or viral elements to direct expression may be engineered. For example, when using adenovirus expression vectors, the imaging agent coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the imaging agent in infected hosts (see, e.g., Logan & Shenk, Proc. Natl. Acad. Sci. USA, 81: 3655-3659, 1984). Alternatively, the vaccinia virus 7.5 K promoter may be used (See, e.g., Mackett et al., Proc. Natl. Acad. Sci. USA, 79: 7415-7419, 1982; Mackett et al., J. Virol. 49: 857-864, 1984; Panicali et al., Proc. Natl. Acad. Sci. USA 79: 4927-4931, 1982).

In some embodiments, vectors based on bovine papilloma virus which have the ability to replicate as extrachromosomal elements may be engineered (Sarver et al., Mol. Cell. Biol. 1: 486, 1981). Shortly after entry of this DNA into cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of the imaging agent gene in host cells (Cone & Mulligan, Proc. Natl. Acad. Sci. USA, 81:6349-6353, 1984). High level expression may also be achieved using inducible promoters, including, but not limited to, the metallothionine IIA promoter and heat shock promoters.

In an illustrative embodiment, stable expression may be used for long-term, high-yield production of recombinant proteins. Rather than using expression vectors which contain viral origins of replication, host cells can be transformed with the imaging agent DNA controlled by appropriate expression control elements (e.g., promoter, enhancer, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including but not limited to, e.g., the herpes simplex virus thymidine kinase (Wigler et al., Cell, 11: 223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy et al., Cell, 22: 817, 1980) genes can be employed in tk-, hgprt- or aprt cells, respectively. Also, antimetabolite resistance can be used as the basis of selection for dhfr, which confers resistance to methotrexate (Wigler et al., Proc. Natl. Acad. Sci. USA, 77: 3567, 1980; O'Hare et al., Proc. Natl. Acad. Sci. USA, 8: 1527, 1981); gpt, which confers resistance to mycophenolic acid (Mulligan & Berg, Proc. Natl. Acad. Sci. USA, 78: 2072, 1981; neo, which confers resistance to the aminoglycoside G-418 (Colberre-Garapin et al., J. Mol. Biol., 150:1, 1981); and hygro, which confers resistance to hygromycin (Santerre et al., Gene, 30: 147, 1984). Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047, 1988); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987).

The construct described herein can also contain a tag to simplify isolation of the imaging agent. For example, a polyhistidine tag of, e.g., six histidine residues, can be incorporated at the amino terminal end of the fluorescent protein. The polyhistidine tag allows convenient isolation of the protein in a single step by nickel-chelate chromatography.

The expression vector can be transfected into a host cell for expression of the recombinant nucleic acid. Host cells can be selected for high levels of expression in order to purify the fluorescent indicator fusion protein. E. coli is useful for this purpose. Alternatively, the host cell can be a prokaryotic or eukaryotic cell selected to image with the imaging agent. The cell can be, e.g., a cultured cell or a cell in vivo.

One advantage of imaging agent is that they are prepared by normal protein biosynthesis. The constructs can be expressed in E. coli in large scale. Purification from bacteria is simplified when the sequences include polyhistidine tags for one-step purification by nickel-chelate chromatography. Alternatively, the substrates can be expressed directly in a desired host cell for assays in situ.

In particular, transcription can be controlled by certain control sequences, for instance promoters and enhancers, that are operably linked to the functional coding nucleic acid sequence. A promoter contains specific DNA sequences that are recognized by transcription factors and other regulatory factors. Transcription factors initiate the transcription of an operably linked nucleic acid. Regulatory factors may be proteins or other chemicals that modulate the rate of transcription. An enhancer is a cis-acting regulatory sequence element that can further modulate the transcription of a nucleic acid sequence.

In some embodiments, the nucleic acid construct encoding an imaging agent includes a promoter operably linked to the nucleic acid encoding the imaging agent. The promoter may be a strong constitutive promoter, a weak constitutive promoter, a tissue-specific promoter, or an inducible promoter. Strong constitutive promoters are very active and thus result in expression of a sufficient amount of protein that is useful for imaging a cell nucleus with strong fluorescence. A weak constitutive promoter is minimally active and thus results in expression of an amount of protein that is useful for clearly showing the position and shape of a cell's condensed chromosomes without interfering background fluorescence. Inducible promoters respond to a stimulus, thus allowing the level of the encoded polypeptide to be controlled over a range of levels for a variety of applications. In illustrative embodiments, a strong constitutive promoter is a CMV early promoter. In illustrative embodiments, a weak constitutive promoter is a truncated CMV early promoter. Examples of inducible promoters are a lac promoter, which can be induced by IPTG, an hsp70 promoter, which can be induced by heat shock, a tetracycline-inducible promoter, an RSL1-inducible promoter, the glucocorticoid-inducible promoter, and other hormone-induced promoters.

The nucleic acid construct encoding the protein imaging agent may be introduced into a vector. Vectors facilitate the cloning, isolation, and manipulation of nucleic acid constructs. Some vectors include sequences that are useful for particular applications, for instance, to control the level of expression of a polypeptide within a cell. In some embodiments, the vectors include, but are not limited to, pGEM-T Easy, pBluescript, TOPO cloning vectors, pCR-Script, and pT7Blue-T. In illustrative embodiments, a vector that includes an imaging agent has the sequence according to SEQ ID NO: 1 or SEQ ID NO: 2.

In another aspect, this disclosure provides a cell which contains an imaging agent, a nucleic acid construct expressing an imaging agent, and/or a vector that contains a nucleic acid construct expressing a fluorescent protein imaging agent. In some embodiments, the imaging agent may be present in various human cancer cell lines. Examples of human cancer cell lines include, but are not limited to, A549, H1299, HeLa, HL60, K562, KG-1, Jurkat, Lncap, MCF-7, MDA-MB-438, T47D, THP-1, U87, SHSY5Y, MCF-10A, T84, Peer, and BxPC3. Other useful human and non-human cell lines known to one skilled in the art may also be used. Cells expressing the fluorescent protein imaging agent may be used to assess the effects of drugs and other agents on cell division, growth, and apoptosis in cancer and other types of cells, for example.

In an illustrative embodiment, the nucleic acid construct is pHMGB1-EGFP, which has a sequence according to SEQ ID NO: 1, as shown below

| FEATURES | Location |
|---|---|
| CMV early promoter | 1 . . . 589 |
| HMGB1 | 639 . . . 1286 |
| EGFP | 1326 . . . 2045 |
| SV40/polyA | 2199 . . . 2249 |
| Kana/Neo | 3276 . . . 4070 |
| PUC origin | 4655 . . . 5298 |

| | |
|---|---|
| 1 | TAGTTATTAA TAGTAATCAA TTACGGGGTC ATTAGTTCAT AGCCCATATA TGGAGTTCCG |
| 61 | CGTTACATAA CTTACGGTAA ATGGCCCGCC TGGCTGACCG CCCAACGACC CCCGCCCATT |
| 121 | GACGTCAATA ATGACGTATG TTCCCATAGT AACGCCAATA GGGACTTTCC ATTGACGTCA |
| 181 | ATGGGTGGAG TATTTACGGT AAACTGCCCA CTTGGCAGTA CATCAAGTGT ATCATATGCC |
| 241 | AAGTACGCCC CCTATTGACG TCAATGACGG TAAATGGCCC GCCTGGCATT ATGCCCAGTA |
| 301 | CATGACCTTA TGGGACTTTC CTACTTGGCA GTACATCTAC GTATTAGTCA TCGCTATTAC |
| 361 | CATGGTGATG CGGTTTTGGC AGTACATCAA TGGGCGTGGA TAGCGGTTTG ACTCACGGGG |
| 421 | ATTTCCAAGT CTCCACCCCA TTGACGTCAA TGGGAGTTTG TTTTGGCACC AAAATCAACG |
| 481 | GGACTTTCCA AAATGTCGTA ACAACTCCGC CCCATTGACG CAAATGGGCG GTAGGCGTGT |
| 541 | ACGGTGGGAG GTCTATATAA GCAGAGCTGG TTTAGTGAAC CGTCAGATCC GCTAGCGCTA |
| 601 | CCGGACTCAG ATCTCGAGCT CAAGCTTCGA ATTCGATTAT GGGCAAAGGA GATCCTAAGA |
| 661 | AGCCGAGAGG CAAAATGTCA TCATATGCAT TTTTTGTGCA AACTTGTCGG GAGGAGCATA |
| 721 | AGAAGAAGCA CCCAGATGCT TCAGTCAACT TCTCAGAGTT TTCTAAGAAG TGCTCAGAGA |
| 781 | GGTGGAAGAC CATGTCTGCT AAAGAGAAAG GAAAATTTGA AGATATGGCA AAAGCGGACA |
| 841 | AGGCCCGTTA TGAAAGAGAA ATGAAAACCT ATATCCCTCC CAAAGGGGAG ACAAAAAAGA |
| 901 | AGTTCAAGGA TCCCAATGCA CCCAAGAGGC CTCCTTCGGC CTTCTTCCTC TTCTGCTCTG |
| 961 | AGTATCGCCC AAAAATCAAA GGAGAACATC CTGGCCTGTC CATTGGTGAT GTTGCGAAGA |
| 1021 | AACTGGGAGA GATGTGGAAT AACACTGCTG CAGATGACAA GCAGCCTTAT GAAAAGAAGG |
| 1081 | CTGCGAAGCT GAAGGAAAAA TACGAAAAGG ATATTGCTGC ATATCGAGCT AAAGGAAAGC |
| 1141 | CTGATGCAGC AAAAAAGGGA GTTGTCAAGG CTGAAAAAAG CAAGAAAAAG AAGGAAGAGG |
| 1201 | AGGAAGATGA GGAAGATGAA GAGGATGAGG AGGAGGAGGA AGATGAAGAA GATGAAGATG |
| 1261 | AAGAAGAAGA TGATGATGAT GAATCGTCGA CGGTACCGCG GGCCCGGGAT CCACCGGTCG |
| 1321 | CCACCATGGT GAGCAAGGGC GAGGAGCTGT TCACCGGGGT GGTGCCCATC CTGGTCGAGC |
| 1381 | TGGACGGCGA CGTAAACGGC CACAAGTTCA GCGTGTCCGG CGAGGGCGAG GGCGATGCCA |
| 1441 | CCTACGGCAA GCTGACCCTG AAGTTCATCT GCACCACCGG CAAGCTGCCC GTGCCCTGGC |
| 1501 | CCACCCTCGT GACCACCCTG ACCTACGGCG TGCAGTGCTT CAGCCGCTAC CCCGACCACA |
| 1561 | TGAAGCAGCA CGACTTCTTC AAGTCCGCCA TGCCCGAAGG CTACGTCCAG GAGCGCACCA |
| 1621 | TCTTCTTCAA GGACGACGGC AACTACAAGA CCCGCGCCGA GGTGAAGTTC GAGGGCGACA |
| 1681 | CCCTGGTGAA CCGCATCGAG CTGAAGGGCA TCGACTTCAA GGAGGACGGC AACATCCTGG |
| 1741 | GGCACAAGCT GGAGTACAAC TACAACAGCC ACAACGTCTA TATCATGGCC GACAAGCAGA |
| 1801 | AGAACGGCAT CAAGGTGAAC TTCAAGATCC GCCACAACAT CGAGGACGGC AGCGTGCAGC |
| 1861 | TCGCCGACCA CTACCAGCAG AACACCCCCA TCGGCGACGG CCCCGTGCTG CTGCCCGACA |
| 1921 | ACCACTACCT GAGCACCCAG TCCGCCCTGA GCAAAGACCC CAACGAGAAG CGCGATCACA |
| 1981 | TGGTCCTGCT GGAGTTCGTG ACCGCCGCCG GGATCACTCT CGGCATGGAC GAGCTGTACA |
| 2041 | AGTAAAGCGG CCGCGACTCT AGATCATAAT CAGCCATACC ACATTTGTAG AGGTTTTACT |
| 2101 | TGCTTTAAAA AACCTCCCAC ACCTCCCCCT GAACCTGAAA CATAAAATGA ATGCAATTGT |
| 2161 | TGTTGTTAAC TTGTTTATTG CAGCTTATAA TGGTTACAAA TAAAGCAATA GCATCACAAA |
| 2221 | TTTCACAAAT AAAGCATTTT TTTCACTGCA TTCTAGTTGT GGTTTGTCCA AACTCATCAA |
| 2281 | TGTATCTTAA GGCGTAAATT GTAAGCGTTA ATATTTTGTT AAAATTCGCG TTAAATTTTT |
| 2341 | GTTAAATCAG CTCATTTTTT AACCAATAGG CCGAAATCGG CAAAATCCCT TATAAATCAA |
| 2401 | AAGAATAGAC CGAGATAGGG TTGAGTGTTG TTCCAGTTTG GAACAAGAGT CCACTATTAA |
| 2461 | AGAACGTGGA CTCCAACGTC AAAGGGCGAA AAACCGTCTA TCAGGGCGAT GGCCCACTAC |
| 2521 | GTGAACCATC ACCCTAATCA AGTTTTTTGG GGTCGAGGTG CCGTAAAGCA CTAAATCGGA |
| 2581 | ACCCTAAAGG GAGCCCCCGA TTTAGAGCTT GACGGGGAAA GCCGGCGAAC GTGGCGAGAA |
| 2641 | AGGAAGGGAA GAAAGCGAAA GGAGCGGGCG CTAGGGCGCT GGCAAGTGTA GCGGTCACGC |
| 2701 | TGCGCGTAAC CACCACACCC GCCGCGCTTA ATGCGCCGCT ACAGGGCGCG TCAGGTGGCA |
| 2761 | CTTTTCGGGG AAATGTGCGC GGAACCCCTA TTTGTTTATT TTTCTAAATA CATTCAAATA |
| 2821 | TGTATCCGCT CATGAGACAA TAACCCTGAT AAATGCTTCA ATAATATTGA AAAAGGAAGA |
| 2881 | GTCCTGAGGC GGAAAGAACC AGCTGTGGAA TGTGTGTCAG TTAGGGTGTG GAAAGTCCCC |
| 2941 | AGGCTCCCCA GCAGGCAGAA GTATGCAAAG CATGCATCTC AATTAGTCAG CAACCAGGTG |
| 3001 | TGGAAAGTCC CCAGGCTCCC CAGCAGGCAG AAGTATGCAA AGCATGCATC TCAATTAGTC |
| 3061 | AGCAACCATA GTCCCGCCCC TAACTCCGCC CATCCCGCCC CTAACTCCGC CCAGTTCCGC |
| 3121 | CCATTCTCCG CCCCATGGCT GACTAATTTT TTTTATTTAT GCAGAGGCCG AGGCCGCCTC |
| 3181 | GGCCTCTGAG CTATTCCAGA AGTAGTGAGG AGGCTTTTTT GGAGGCCTAG GCTTTTGCAA |
| 3241 | AGATCGATCA AGAGACAGGA TGAGGATCGT TTCGCATGAT TGAACAAGAT GGATTGCACG |
| 3301 | CAGGTTCTCC GGCCGCTTGG GTGGAGAGGC TATTCGGCTA TGACTGGGCA CAACAGACAA |
| 3361 | TCGGCTGCTC TGATGCCGCC GTGTTCCGGC TGTCAGCGCA GGGGCGCCCG GTTCTTTTTG |
| 3421 | TCAAGACCGA CCTGTCCGGT GCCCTGAATG AACTGCAAGA CGAGGCAGCG CGGCTATCGT |
| 3481 | GGCTGGCCAC GACGGGCGTT CCTTGCGCAG CTGTGCTCGA CGTTGTCACT GAAGCGGGAA |
| 3541 | GGGACTGGCT GCTATTGGGC GAAGTGCCGG GGCAGGATCT CCTGTCATCT CACCTTGCTC |
| 3601 | CTGCCGAGAA AGTATCCATC ATGGCTGATG CAATGCGGCG GCTGCATACG CTTGATCCGG |
| 3661 | CTACCTGCCC ATTCGACCAC CAAGCGAAAC ATCGCATCGA GCGAGCACGT ACTCGGATGG |
| 3721 | AAGCCGGTCT TGTCGATCAG GATGATCTGG ACGAAGAGCA TCAGGGGCTC GCGCCAGCCG |
| 3781 | AACTGTTCGC CAGGCTCAAG GCGAGCATGC CCGACGGCGA GGATCTCGTC GTGACCCATG |
| 3841 | GCGATGCCTG CTTGCCGAAT ATCATGGTGG AAAATGGCCG CTTTTCTGGA TTCATCGACT |
| 3901 | GTGGCCGGCT GGGTGTGGCG GACCGCTATC AGGACATAGC GTTGGCTACC CGTGATATTG |
| 3961 | CTGAAGAGCT TGGCGGCGAA TGGGCTGACC GCTTCCTCGT GCTTTACGGT ATCGCCGCTC |
| 4021 | CCGATTCGCA GCGCATCGCC TTCTATCGCC TTCTTGACGA GTTCTTCTGA GCGGGACTCT |
| 4081 | GGGGTTCGAA ATGACCGACC AAGCGACGCC CAACCTGCCA TCACGAGATT TCGATTCCAC |
| 4141 | CGCCGCCTTC TATGAAAGGT TGGGCTTCGG AATCGTTTTC CGGGACGCCG GCTGGATGAT |
| 4201 | CCTCCAGCGC GGGGATCTCA TGCTGGAGTT CTTCGCCCAC CCTAGGGGGA GGCTAACTGA |
| 4261 | AACACGGAAG GAGACAATAC CGGAAGGAAC CCGCGCTATG ACGGCAATAA AAAGACAGAA |
| 4321 | TAAAACGCAC GGTGTTGGGT CGTTTGTTCA TAAACGCGGG GTTCGGTCCC AGGGCTGGCA |
| 4381 | CTCTGTCGAT ACCCCACCGA GACCCCATTG GGGCCAATAC GCCCGCGTTT CTTCCTTTTC |
| 4441 | CCCACCCCAC CCCCCAAGTT CGGGTGAAGG CCCAGGGCTC GCAGCCAACG TCGGGGCGGC |
| 4501 | AGGCCCTGCC ATAGCCTCAG GTTACTCATA TATACTTTAG ATTGATTTAA AACTTCATTT |
| 4561 | TTAATTTAAA AGGATCTAGG TGAAGATCCT TTTTGATAAT CTCATGACCA AAATCCCTTA |
| 4621 | ACGTGAGTTT TCGTTCCACT GAGCGTCAGA CCCCGTAGAA AAGATCAAAG GATCTTCTTG |
| 4681 | AGATCCTTTT TTTCTGCGCG TAATCTGCTG CTTGCAAACA AAAAAACCAC CGCTACCAGC |
| 4741 | GGTGGTTTGT TTGCCGGATC AAGAGCTACC AACTCTTTTT CCGAAGGTAA CTGGCTTCAG |
| 4801 | CAGAGCGCAG ATACCAAATA CTGTCCTTCT AGTGTAGCCG TAGTTAGGCC ACCACTTCAA |
| 4861 | GAACTCTGTA GCACCGCCTA CATACCTCGC TCTGCTAATC CTGTTACCAG TGGCTGCTGC |
| 4921 | CAGTGGCGAT AAGTCGTGTC TTACCGGGTT GGACTCAAGA CGATAGTTAC CGGATAAGGC |
| 4981 | GCAGCGGTCG GGCTGAACGG GGGGTTCGTG CACACAGCCC AGCTTGGAGC GAACGACCTA |
| 5041 | CACCGAACTG AGATACCTAC AGCGTGAGCT ATGAGAAAGC GCCACGCTTC CCGAAGGGAG |
| 5101 | AAAGGCGGAC AGGTATCCGG TAAGCGGCAG GGTCGGAACA GGAGAGCGCA CGAGGGAGCT |
| 5161 | TCCAGGGGGA AACGCCTGGT ATCTTTATAG TCCTGTCGGG TTTCGCCACC TCTGACTTGA |
| 5221 | GCGTCGATTT TTGTGATGCT CGTCAGGGGG GCGGAGCCTA TGGAAAAACG CCAGCAACGC |
| 5281 | GGCCTTTTTA CGGTTCCTGG CCTTTTGCTG GCCTTTTGCT CACATGTTCT TTCCTGCGTT |
| 5341 | ATCCCCTGAT TCTGTGGATA ACCGTATTAC CGCCATGCAT |

In an illustrative embodiment, the nucleic acid construct is ptrEGFP-HMGB1, which has a sequence according to SEQ ID NO: 2, as shown below:

| FEATURES | Location |
|---|---|
| Truncated CMV promoter | 3 . . . 92 |
| HMGB1 | 142 . . . 789 |
| EGFP | 829 . . . 1548 |
| SV40/polyA | 1702 . . . 1752 |
| Kana/Neo | 3809 . . . 3827 |
| PUC origin | 4158 . . . 4801 |

| | |
|---|---|
| 1 | GCAACAACTC CGCCCCATTG ACGCAAATGG GCGGTAGGCG TGTACGGTGG GAGGTCTATA |
| 61 | TAAGCAGAGC TGGTTTAGTG AACCGTCAGA TCCGCTAGCG CTACCGGACT CAGATCTCGA |
| 121 | GCTCAAGCTT CGAATTCGAT TATGGGCAAA GGAGATCCTA AGAAGCCGAG AGGCAAAATG |
| 181 | TCATCATATG CATTTTTTGT GCAAACTTGT CGGGAGGAGC ATAAGAAGAA GCACCCAGAT |
| 241 | GCTTCAGTCA ACTTCTCAGA GTTTTCTAAG AAGTGCTCAG AGAGGTGGAA GACCATGTCT |
| 301 | GCTAAAGAGA AAGGAAAATT TGAAGATATG GCAAAAGCGG ACAAGGCCCG TTATGAAAGA |
| 361 | GAAATGAAAA CCTATATCCC TCCCAAAGGG GAGACAAAAA AGAAGTTCAA GGATCCCAAT |
| 421 | GCACCCAAGA GGCCTCCTTC GGCCTTCTTC CTCTTCTGCT CTGAGTATCG CCCAAAAATC |
| 481 | AAAGGAGAAC ATCCTGGCCT GTCCATTGGT GATGTTGCGA AGAAACTGGG AGAGATGTGG |
| 541 | AATAACACTG CTGCAGATGA CAAGCAGCCT TATGAAAAGA AGGCTGCGAA GCTGAAGGAA |
| 601 | AAATACGAAA AGGATATTGC TGCATATCGA GCTAAAGGAA AGCCTGATGC AGCAAAAAAG |
| 661 | GGAGTTGTCA AGGCTGAAAA AAGCAAGAAA AAGAAGGAAG AGGAGGAAGA TGAGGAAGAT |
| 721 | GAAGAGGATG AGGAGGAGGA GGAAGATGAA GAAGATGAAG ATGAAGAAGA AGATGATGAT |
| 781 | GATGAATCGT CGACGGTACC GCGGGCCCGG GATCCACCGG TCGCCACCAT GGTGAGCAAG |
| 841 | GGCGAGGAGC TGTTCACCGG GGTGGTGCCC ATCCTGGTCG AGCTGGACGG CGACGTAAAC |
| 901 | GGCCACAAGT TCAGCGTGTC CGGCGAGGGC GAGGGCGATG CCACCTACGG CAAGCTGACC |
| 961 | CTGAAGTTCA TCTGCACCAC CGGCAAGCTG CCCGTGCCCT GGCCCACCCT CGTGACCACC |
| 1021 | CTGACCTACG GCGTGCAGTG CTTCAGCCGC TACCCCGACC ACATGAAGCA GCACGACTTC |
| 1081 | TTCAAGTCCG CCATGCCCGA AGGCTACGTC CAGGAGCGCA CCATCTTCTT CAAGGACGAC |
| 1141 | GGCAACTACA AGACCCGCGC CGAGGTGAAG TTCGAGGGCG ACACCCTGGT GAACCGCATC |
| 1201 | GAGCTGAAGG GCATCGACTT CAAGGAGGAC GGCAACATCC TGGGGCACAA GCTGGAGTAC |
| 1261 | AACTACAACA GCCACAACGT CTATATCATG GCCGACAAGC AGAAGAACGG CATCAAGGTG |
| 1321 | AACTTCAAGA TCCGCCACAA CATCGAGGAC GGCAGCGTGC AGCTCGCCGA CCACTACCAG |
| 1381 | CAGAACACCC CCATCGGCGA CGGCCCCGTG CTGCTGCCCG ACAACCACTA CCTGAGCACC |
| 1441 | CAGTCCGCCC TGAGCAAAGA CCCCAACGAG AAGCGCGATC ACATGGTCCT GCTGGAGTTC |
| 1501 | GTGACCGCCG CCGGGATCAC TCTCGGCATG GACGAGCTGT ACAAGTAAAG CGGCCGCGAC |
| 1561 | TCTAGATCAT AATCAGCCAT ACCACATTTG TAGAGGTTTT ACTTGCTTTA AAAAACCTCC |
| 1621 | CACACCTCCC CCTGAACCTG AAACATAAAA TGAATGCAAT TGTTGTTGTT AACTTGTTTA |
| 1681 | TTGCAGCTTA TAATGGTTAC AAATAAAGCA ATAGCATCAC AAATTTCACA AATAAAGCAT |
| 1741 | TTTTTTCACT GCATTCTAGT TGTGGTTTGT CCAAACTCAT CAATGTATCT TAAGGCGTAA |
| 1801 | ATTGTAAGCG TTAATATTTT GTTAAAATTC GCGTTAAATT TTTGTTAAAT CAGCTCATTT |
| 1861 | TTTAACCAAT AGGCCGAAAT CGGCAAAATC CCTTATAAAT CAAAAGAATA GACCGAGATA |
| 1921 | GGGTTGAGTG TTGTTCCAGT TTGGAACAAG AGTCCACTAT TAAAGAACGT GGACTCCAAC |
| 1981 | GTCAAAGGGC GAAAAACCGT CTATCAGGGC GATGGCCCAC TACGTGAACC ATCACCCTAA |
| 2041 | TCAAGTTTTT TGGGGTCGAG GTGCCGTAAA GCACTAAATC GGAACCCTAA AGGGAGCCCC |
| 2101 | CGATTTAGAG CTTGACGGGG AAAGCCGGCG AACGTGGCGA GAAAGGAAGG GAAGAAAGCG |
| 2161 | AAAGGAGCGG GCGCTAGGGC GCTGGCAAGT GTAGCGGTCA CGCTGCGCGT AACCACCACA |
| 2221 | CCCGCCGCGC TTAATGCGCC GCTACAGGGC GCGTCAGGTG GCACTTTTCG GGGAAATGTG |
| 2281 | CGCGGAACCC CTATTTGTTT ATTTTTCTAA ATACATTCAA ATATGTATCC GCTCATGAGA |
| 2341 | CAATAACCCT GATAAATGCT TCAATAATAT TGAAAAAGGA AGAGTCCTGA GGCGGAAAGA |
| 2401 | ACCAGCTGTG GAATGTGTGT CAGTTAGGGT GTGGAAAGTC CCCAGGCTCC CCAGCAGGCA |
| 2461 | GAAGTATGCA AAGCATGCAT CTCAATTAGT CAGCAACCAG GTGTGGAAAG TCCCCAGGCT |
| 2521 | CCCCAGCAGG CAGAAGTATG CAAAGCATGC ATCTCAATTA GTCAGCAACC ATAGTCCCGC |
| 2581 | CCCTAACTCC GCCCATCCCG CCCCTAACTC CGCCCAGTTC CGCCCATTCT CCGCCCCATG |
| 2641 | GCTGACTAAT TTTTTTTATT TATGCAGAGG CCGAGGCCGC CTCGGCCTCT GAGCTATTCC |
| 2701 | AGAAGTAGTG AGGAGGCTTT TTTGGAGGCC TAGGCTTTTG CAAAGATCGA TCAAGAGACA |
| 2761 | GGATGAGGAT CGTTTCGCAT GATTGAACAA GATGGATTGC ACGCAGGTTC TCCGGCCGCT |
| 2821 | TGGGTGGAGA GGCTATTCGG CTATGACTGG GCACAACAGA CAATCGGCTG CTCTGATGCC |
| 2881 | GCCGTGTTCC GGCTGTCAGC GCAGGGGCGC CCGGTTCTTT TTGTCAAGAC CGACCTGTCC |
| 2941 | GGTGCCCTGA ATGAACTGCA AGACGAGGCA GCGCGGCTAT CGTGGCTGGC CACGACGGGC |
| 3001 | GTTCCTTGCG CAGCTGTGCT CGACGTTGTC ACTGAAGCGG GAAGGGACTG GCTGCTATTG |
| 3061 | GGCGAAGTGC CGGGGCAGGA TCTCCTGTCA TCTCACCTTG CTCCTGCCGA GAAAGTATCC |
| 3121 | ATCATGGCTG ATGCAATGCG GCGGCTGCAT ACGCTTGATC CGGCTACCTG CCCATTCGAC |
| 3181 | CACCAAGCGA AACATCGCAT CGAGCGAGCA CGTACTCGGA TGGAAGCCGG TCTTGTCGAT |
| 3241 | CAGGATGATC TGGACGAAGA GCATCAGGGG CTCGCGCCAG CCGAACTGTT CGCCAGGCTC |
| 3301 | AAGGCGAGCA TGCCCGACGG CGAGGATCTC GTCGTGACCC ATGGCGATGC CTGCTTGCCG |
| 3361 | AATATCATGG TGGAAAATGG CCGCTTTTCT GGATTCATCG ACTGTGGCCG GCTGGGTGTG |
| 3421 | GCGGACCGCT ATCAGGACAT AGCGTTGGCT ACCCGTGATA TTGCTGAAGA GCTTGGCGGC |
| 3481 | GAATGGGCTG ACCGCTTCCT CGTGCTTTAC GGTATCGCCG CTCCCGATTC GCAGCGCATC |
| 3541 | GCCTTCTATC GCCTTCTTGA CGAGTTCTTC TGAGCGGGAC TCTGGGGTTC GAAATGACCG |
| 3601 | ACCAAGCGAC GCCCAACCTG CCATCACGAG ATTTCGATTC CACCGCCGCC TTCTATGAAA |
| 3661 | GGTTGGGCTT CGGAATCGTT TTCCGGGACG CCGGCTGGAT GATCCTCCAG CGCGGGGATC |
| 3721 | TCATGCTGGA GTTCTTCGCC CACCCTAGGG GGAGGCTAAC TGAAACACGG AAGGAGACAA |
| 3781 | TACCGGAAGG AACCCGCGCT ATGACGGCAA TAAAAAGACA GAATAAAACG CACGGTGTTG |
| 3841 | GGTCGTTTGT TCATAAACGC GGGGTTCGGT CCCAGGGCTG GCACTCTGTC GATACCCCAC |
| 3901 | CGAGACCCCA TTGGGGCCAA TACGCCCGCG TTTCTTCCTT TTCCCCACCC CACCCCCCAA |
| 3961 | GTTCGGGTGA AGGCCCAGGG CTCGCAGCCA ACGTCGGGGC GGCAGGCCCT GCCATAGCCT |
| 4021 | CAGGTTACTC ATATATACTT TAGATTGATT TAAAACTTCA TTTTTAATTT AAAAGGATCT |
| 4081 | AGGTGAAGAT CCTTTTTGAT AATCTCATGA CCAAAATCCC TTAACGTGAG TTTTCGTTCC |
| 4141 | ACTGAGCGTC AGACCCCGTA GAAAAGATCA AAGGATCTTC TTGAGATCCT TTTTTTCTGC |
| 4201 | GCGTAATCTG CTGCTTGCAA ACAAAAAAAC CACCGCTACC AGCGGTGGTT TGTTTGCCGG |
| 4261 | ATCAAGAGCT ACCAACTCTT TTTCCGAAGG TAACTGGCTT CAGCAGAGCG CAGATACCAA |
| 4321 | ATACTGTCCT TCTAGTGTAG CCGTAGTTAG GCCACCACTT CAAGAACTCT GTAGCACCGC |
| 4381 | CTACATACCT CGCTCTGCTA ATCCTGTTAC CAGTGGCTGC TGCCAGTGGC GATAAGTCGT |
| 4441 | GTCTTACCGG GTTGGACTCA AGACGATAGT TACCGGATAA GGCGCAGCGG TCGGGCTGAA |
| 4501 | CGGGGGGTTC GTGCACACAG CCCAGCTTGG AGCGAACGAC CTACACCGAA CTGAGATACC |
| 4561 | TACAGCGTGA GCTATGAGAA AGCGCCACGC TTCCCGAAGG GAGAAAGGCG GACAGGTATC |
| 4621 | CGGTAAGCGG CAGGGTCGGA ACAGGAGAGC GCACGAGGGA GCTTCCAGGG GGAAACGCCT |
| 4681 | GGTATCTTTA TAGTCCTGTC GGGTTTCGCC ACCTCTGACT TGAGCGTCGA TTTTTGTGAT |
| 4741 | GCTCGTCAGG GGGGCGGAGC CTATGGAAAA ACGCCAGCAA CGCGGCCTTT TTACGGTTCC |
| 4801 | TGGCCTTTTG CTGGCCTTTT GCTCACATGT TCTTTCCTGC GTTATCCCCT GATTCTGTGG |
| 4861 | ATAACCGTAT TACCGCCATG CAT |

### I. Methods for Live Cell Nuclear and Chromosome Imaging

A. Methods for Monitoring the Imaging Agent in Live Cells

In another aspect, this disclosure provides a method for imaging live cells by introducing into cells an imaging agent, a nucleic acid construct expressing an imaging agent, and/or a vector that contains a nucleic acid construct expressing an imaging agent, and then monitoring the imaging agent in live cells. In some embodiments, the method may be used to visualize the shape, size, position, and/or state of the cell nucleus or chromosomes. Observing the state of the nucleus and chromosomes is useful for monitoring cellular phenomena because particular structures and/or arrangements of the nucleus and chromosomes are indicative of certain cellular phenomena, such as apoptosis, cell division, mitosis, meiosis, carcinogenesis, and the effects of a therapeutic or other agent that may or may not be toxic to the cell. For instance, cell division is marked by the positioning of condensed chromosomes along the mitotic spindle near the center of a cell. Apoptosis is marked by a distinct cell morphology that includes cell rounding, the fragmentation of the nucleus, and the condensation and fragmentation of chromosomes.

In some embodiments, the imaging agent may be introduced into cells directly. Some methods for direct introduction of proteins and peptides are microinjection, cell-penetrating peptide, transfection, bombardment, liposomes, lipofection, cell membrane permeabilization, freeze/thaw, heat shock, nucleofection, electroporation, electrostatic adsorption, and receptor-mediated endocytosis. In other embodiments, the imaging agent is expressed from a nucleic acid construct introduced into cells. Some methods for introducing nucleic acid constructs into cells are microinjection, transformation, transduction, transfection, bombardment (“gene gun”), liposomes, lipofection, cell membrane permeabilization, freeze/thaw, heat shock, nucleofection, electroporation, electrostatic adsorption, and receptor-mediated endocytosis. The nucleic acid may be integrated into the chromosome, may be present as an independently-replicating plasmid, or may be transiently expressed. In all cases, expression of the imaging agent may be controlled by a strong constitutive promoter, a weak constitutive promoter, a tissue-specific promoter, or an inducible promoter.

In one embodiment, the imaging agent is used to generate an image of the chromosomes and/or nucleus of a cell. In one embodiment, the image is observed by the user directly using a fluorescence microscope. In another embodiment, the image is represented by individual photons emitted by the fluorescence domain following excitation at the appropriate wavelength. By accumulating these detected photons in a digital image processor over time, an image can be acquired and constructed. At least two types of photodetector devices can detect individual photons and generate a signal which can be analyzed by an image processor. Reduced-noise photodetection devices achieve sensitivity by reducing the background noise in the photon detector, as opposed to amplifying the photon signal. Noise is reduced primarily by cooling the detector array. The devices include charge coupled device (CCD) cameras referred to as “backthinned,” cooled CCD cameras. “Backthinned” refers to an ultra-thin backplate that reduces the path length that a photon follows to be detected, thereby increasing the quantum efficiency. Photon amplification devices amplify photons before they hit the detection screen. This class includes CCD cameras with intensifiers, such as microchannel intensifiers. A microchannel intensifier may contain a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera. Image processors process signals generated by photodetector devices which count photons in order to construct an image which can be, for example, displayed on a monitor or printed on a video printer. Once the images are in the form of digital files, they can be manipulated by a variety of image processing programs and printed.

In one embodiment, the number of photons emitted by the imaging agent are counted. If a photon counting approach is used, the measurement of photon emission generates an array of numbers, representing the number of photons detected at each pixel location, in the image processor. These numbers are used to generate an image by normalizing the photon counts (either to a fixed, pre-selected value, or to the maximum number detected in any pixel) and converting the normalized number to a brightness (greyscale) or to a color (pseudocolor) that is displayed on a monitor. In a pseudocolor representation, typical color assignments are as follows. Pixels with zero photon counts are assigned black, low counts blue, and increasing counts colors of increasing wavelength, on up to red for the highest photon count values. The location of colors on the monitor represents the distribution of photon emission, and, accordingly, the location of light-generating imaging agents.

In order to provide a frame of reference for the nucleus and/or chromosomes in a cell, a full color or greyscale image of the cell(s) from which photon emission was measured may be constructed, for example, by opening a door to the imaging chamber, or box, in dim room light, and measuring reflected photons. The full color or greyscale image may be constructed either before measuring photon emission, or after. The image of photon emission is superimposed on the full color or greyscale image to produce a composite image of photon emission in relation to the cell(s).

If it is desired to follow the localization and/or the signal from an imaging agent over time, for example, to record the effects of a treatment on the distribution and/or localization of the imaging agent, the measurement of photon emission, or imaging can be repeated at selected time intervals to construct a series of images. The intervals can be as short as minutes, or as long as days or weeks.

Images generated by methods and/or using the imaging agents described herein may be analyzed by a variety of methods. They range from a simple visual examination, mental evaluation and/or printing of a hardcopy, to sophisticated digital image analysis. Interpretation of the information obtained from an analysis depends on the phenomenon under observation and the entity being used.

In some embodiments, the difference between the fluorescence level of a cell comprising the imaging agent compared to a reference fluorescence level is indicative of the cellular phenomenon undergoing study. The reference level may be a control cell. Depending on the type of investigation, those of skill in the art are able to select appropriate controls. In an illustrative embodiment, where the cellular phenomenon causes loss of integrity of the nucleus or chromosomes of a cell, the difference in the fluorescence level may be a decrease in the fluorescence intensity compared to a reference fluorescence level. In another illustrative embodiment, where the cellular phenomenon causes loss of integrity of the nucleus or chromosomes of a cell, the difference fluorescence level may be a change in the localization of the fluorescence compared the localization of the fluorescence within the reference cell.

B. Methods for Screening Molecular Agents

The methods may be practiced in vivo wherein a test compound (e.g., a biological effector molecule) and the imaging agent (or a vector encoding the same) are contacted to a cell sample under conditions to allow detection of the imaging agent in the cells. The disclosure thus also pertains to the field of predictive medicine in which diagnostic assays, prognostic assays, pharmacogenomics, and monitoring clinical trials are used for prognostic (predictive) purposes to analyze the effects of a biological effector molecule on a sample of cells obtained from a subject.

In some embodiments, the state of the nucleus and chromosomes in the presence and absence of a biological effector molecule (a drug or other molecular agent) is compared. In one embodiment, the methods provide for monitoring the influence of agents on one or more nucleus-associated characteristics in a cell. Such assays can be applied in basic drug screening and in clinical trials. For example, the effectiveness of an agent to increase (or decrease) one or more nucleus-associated characteristics can be monitored in clinical trials of subjects exhibiting a medical condition associated with one or more nucleus-associated characteristics. An agent that affects one or more nucleus-associated changes can be identified by administering the agent and observing a response (e.g., a change in the amount or localization of fluorescence). In this way, the one or more nucleus-associated changes can serve as a marker, indicative of the physiological response of the subject to the agent. Accordingly, this response state may be determined before, and at various points during, treatment of the individual with the agent.

In one embodiment, a cell or a population of cells may be administered an agent prior to, simultaneously, or subsequently with the imaging agent or a nucleic acid encoding the imaging agent. The cell or population of cells may then be imaged to ascertain the total level of fluorescence and/or the localization of fluorescence. In either case, the data may be quantified by counting the number of pixels in an image (or at a particular location within the image). The images and/or the quantitative pixel data may be compared to a reference or control cell. The reference cell may be a cell of the same type that has not been contacted with the agent.

In one embodiment, an imaging agent is used to observe the effects of a biological effector molecule on cancer cells. For example, cancer cells typically lose the capacities to undergo apoptosis and to undergo controlled cell division and growth, events that can be followed by the location and characteristics of the nucleus and chromosomes as visualized by the imaging agent. In other embodiments, the effects of compounds including, but not limited to, pesticides; herbicides; small molecules; toxins; nucleic acids; and polypeptides on cells are observed using the imaging agents disclosed herein. Details on the types of biological effector molecules that may be tested are provided below.

The disclosure further provides a method for testing a compound for biological activity in the cells of the test sample. The method (also referred to herein as a “screening assay”) can be used for identifying modulators, i. e., candidate or test compounds or agents (e.g., peptides, peptidomimetics, small molecules or other drugs) that promote effect one or more cellular states, e.g., apoptosis, energetics, metabolism, chromosome structure or dynamics, or cytoskeletal organization. The disclosure also includes compounds identified in the screening assays described herein.

In one embodiment, a combinatorial library of test compounds is used in conjunction with the imaging agent to assess the effects of the compounds on one or more cells. The test compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

Libraries of chemical and/or biological mixtures, such as fungal, bacterial, or algal extracts, are known in the art and can be screened with any of the assays described herein. Examples of methods for the synthesis of molecular libraries can be found in the art, for example in: DeWitt et al., 1993. Proc. Natl. Acad. Sci. U.S.A. 90: 6909; Erb et al., 1994. Proc. Natl. Acad. Sci. U.S.A. 91: 11422; Zuckermann et al., 1994. J. Med. Chem. 37: 2678; Cho et al., 1993. Science 261: 1303; Carrell et al., 1994. Angew. Chem. Int. Ed. Engl. 33: 2059; Carell et al., 1994. Angew. Chem. Int. Ed. Engl. 33: 2061; and Gallop et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, Nature 364: 555-556, 1993), bacteria (Ladner, U.S. Pat. No. 5,223,409), spores (Ladner, U.S. Pat. No. 5,233,409), plasmids (Cull et al., Proc. Natl. Acad. Sci. USA 89: 1865-1869, 1992) or on phage (Scott and Smith, Science 249: 386-390, 1990); Devlin, Science 249: 404-406, 1990; Cwirla et al., Proc. Natl. Acad. Sci. U.S.A. 87: 6378-6382, 1990; Felici, J. Mol. Biol. 222: 301-310, 1991; Ladner, U.S. Pat. No. 5,233,409).

Test compounds as used in the inventive method may be provided from any known compound library, such as small molecule compound libraries, containing inorganic and organic compounds, peptides, proteins, hormones, antibodies, etc. Alternatively, test compounds may be derived from any biological source, such as plants, tissues, body fluids, such as blood, lymph, etc. If the modulatory potential of test compounds from biological sources is analyzed, these sources may be homogenized prior to addition to the cells. Thereby, the test compound is added to the cells in a defined and reproducible manner. Such homogenized sources may be cell suspensions and may contain cells, cell fragments, etc. If the homogenized material is not to be added as such, test compounds may be isolated or extracted from these homogenized sources prior to (or eventually subsequent to) addition of the cells by conventional biochemical methods, such as chromatography, e.g., affinity chromatography (HPLC, FPLC, etc.), size exclusion chromatography, etc., as well as by cell sorting assays, antibody detection, etc.

In one embodiment, just one test compound is contacted to the cells. However, more than one test compound may be added, e.g., 2-10, 2-50, 2-100 or more test compound species added to the sample. This embodiment allows several test compound species to be screened simultaneously.

Detection of the (altered) fluorescence signal(s) of the imaging agent in the host cell is carried out by any of the aforementioned methods for detecting fluorescence. An effect of the test compound on a cell may be shown by observing a shift of fluorescence signal intensity or localization in the cells contacted with the agent when compared to the fluorescence measurement in cells without addition of test compound.

In certain embodiments, the effect of chemotherapeutic agents on one or more cells may be investigated using the imaging agents described herein. Agents or factors may include any chemical compound that induces DNA damage when applied to a cell. Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), famesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference,” Goodman & Gilman's “The Pharmacological Basis of Therapeutics” and in “Remington's Pharmaceutical Sciences,” incorporated herein by reference in relevant parts), and may be combined with the imaging agent in light of the disclosures herein. Some variation in dosage will necessarily occur depending on the condition or types of cells being analyzed. The person responsible for administration will, in any event, determine the appropriate dose. Examples of specific chemotherapeutic agents are described herein. Of course, all of these agents are exemplary rather than limiting, and other agents may be used by a skilled artisan for a specific patient or application. The skilled artisan is directed to “Remington's Pharmaceutical Sciences” 15th Edition, chapter 33, and in particular to pages 624-652. Some variation in dosage will necessarily occur depending on the condition of the subject being treated. The person responsible for administration will, in any event, determine the appropriate dose for the individual subject.

Alkylating agents are drugs that directly interact with genomic DNA to prevent the cancer cell from proliferating. This category of chemotherapeutic drugs represents agents that affect all phases of the cell cycle, that is, they are not phase-specific. An alkylating agent, may include, but is not limited to, a nitrogen mustard, an ethylenimene, a methylmelamine, an alkyl sulfonate, a nitrosourea or a triazines. They include but are not limited to: busulfan, chlorambucil, cisplatin, cyclophosphamide (cytoxan), dacarbazine, ifosfamide, mechlorethamine (mustargen), and melphalan.

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of antitumor antibiotics include, but are not limited to, bleomycin, dactinomycin, daunorubicin, doxorubicin (Adriamycin), plicamycin (mithramycin) and idarubicin.

Corticosteroid hormones are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer.

Some chemotherapy agents do not fall into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they may be used within the compositions and methods described herein.

An anthracenedione such as mitoxantrone has been used for treating acute granulocytic leukemia and breast cancer. A substituted urea such as hydroxyurea has been used in treating chronic granulocytic leukemia, polycythemia vera, essental thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists. These different family members have been shown to either possess similar functions to Bcl-2 or counteract Bcl-2 function and promote cell death (e.g., Bax, Bak, Bik, Bim, Bid, Bad, Harakiri). Non-limiting examples of pro-apoptosis agents include gramicidin, magainin, mellitin, defensin, and cecropin.

In certain embodiments, the molecular agent is an angiogenic agent, such as angiotensin, laminin peptides, fibronectin peptides, plasminogen activator inhibitors, tissue metalloproteinase inhibitors, interferons, interleukin 12, platelet factor 4, IP-10, Gro-beta, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline.

### EXAMPLES

The present disclosure is further illustrated by the following examples, which should not be construed as limiting in any way.

### Example 1

### Preparation of the HMGB1-EGFP Fusion Protein Construct

The HMGB1 cDNA sequence was assembled from genomic DNA using the “genomic DNA splicing” strategy (An et al., PLoS ONE 2(11): e1179 (2007)) and the following primers:

TABLE 1
PCR Primers

| | | | | |
|---|---|---|---|---|
| Exon | Forward | SEQ ID NO: | Reverse | SEQ ID NO: |

Human HMGB1 exon primers (5′→3′)

| | | | | |
|---|---|---|---|---|
| 2 | CTGATTTTACGGAGGTTGATGTC | SEQ ID NO: 3 | TCCTTTCTCTTTAGCAGACATGGT | SEQ ID NO: 4 |
| 3 | AGGTAAGAGGGCTTAAAACATGCTA | SEQ ID NO: 5 | CCCACCCAACAGGAATTTTATACTA | SEQ ID NO: 6 |
| 4 | TATAGTATTTGCACCCTGTCCAATG | SEQ ID NO: 7 | ACCCTAATTTATTTGGTCCTCTGC | SEQ ID NO: 8 |
| 5 | GGATCTACAGATACGTGATATTTTGG | SEQ ID NO: 9 | GACAGGGCTATCTAAAGACACATTC | SEQ ID NO: 10 |

Human HMGB1 overlapping primers (5′→3′)

| | | | | |
|---|---|---|---|---|
| 2 | ATGGGCAAAGGAGATCCT | SEQ ID NO: 11 | AGCAGACATGGTCTTCCACCTCTCTGAGCA | SEQ ID NO: 12 |
| 3 | GGAAGACCATGTCTGCTAAAGA | SEQ ID NO: 13 | CTTGGGTGCATTGGGAT | SEQ ID NO: 14 |
| 4 | CCCAATGCACCCAAGAGGCCTCCTTCGGCCTTCTTCCT | SEQ ID NO: 15 | GCAGCAATATCCTTTTCGTATTTTTCCTTCA | SEQ ID NO: 16 |
| 5 | ACGAAAAGGATATTGCTGCATATCGA | SEQ ID NO: 17 | TGCGCTAGAACCAACTTA | SEQ ID NO: 18 |

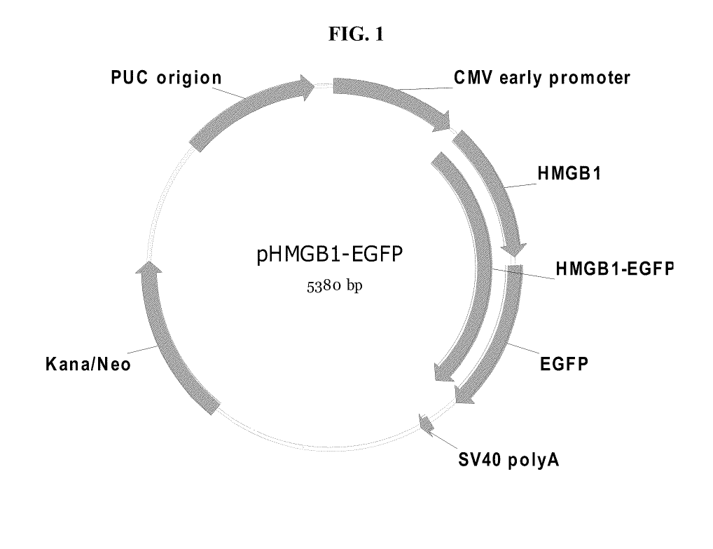

After cloning HMGB1 into pGEM-T Easy (Promega), three clones were sequenced and compared to the wild-type sequence available in GenBank (NM_{—}002128). One wild-type clone was subcloned into a eukaryotic expression vector pEGFP-lac, resulting in a 5380 bp construct pHMGB1-EGFP in which the HMGB1 gene was fused with EGFP and expressed by the CMV early promoter and enhancer (FIG. 1).

### Example 2

### Imaging the Nucleus of Living Cells

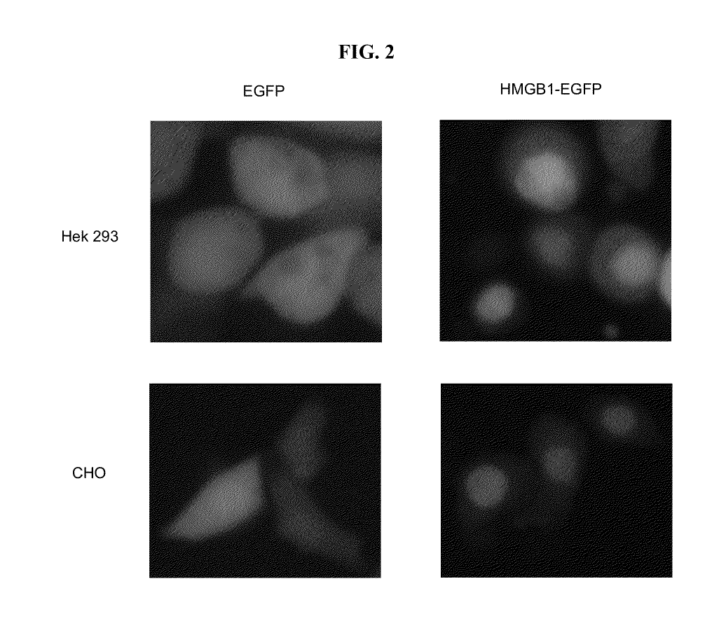

Human embryonic kidney cells (HEK 293FT) and Chinese hamster ovary (CHO) cells were transfected with the pHMGB1-EGFP plasmid prepared as described in Example 1. In parallel control experiments, cells were transfected with plasmid expressing only EGFP. After forty-eight hours, the cells were observed using fluorescence microscopy. As demonstrated by the fluorescence microscope images of FIG. 2, the HMGB1-EGFP fusion protein localized to the nucleus of both cell types, thus allowing for its visualization. However, in control experiments, EGFP alone diffused throughout the cells and did not allow distinguishable imaging of the nucleus. Accordingly, the fusion protein constructs described herein are useful in methods for imaging the nucleus of living cells.

### Example 3

### Monitoring Apoptosis in Living Cells

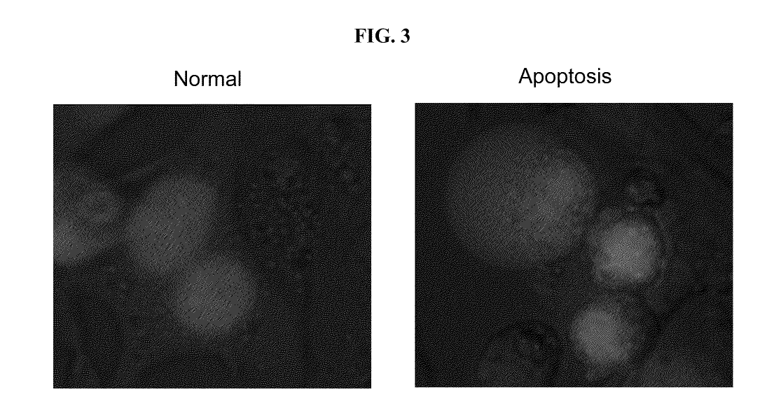

Human embryonic kidney cells (HEK 293FT) and Chinese hamster ovary (CHO) cells were transfected with the HMGB1-EGFP plasmid prepared as described in Example 1. In parallel control experiments, cells were transfected with plasmid expressing only EGFP. After 24 hours, apoptosis was induced by removing fetal bovine serum (FBS) from the cell cultures. FBS was not removed from control cultures. After an additional 48 hours of incubation, the cells were observed by fluorescence microscopy. As shown in the fluorescence microscope images of FIG. 3, removing FBS caused a distinctive round cell shape associated with apoptosis and diffusion of fluorescence throughout the cells as a result of apoptotic disruption of the nucleus. The control cells maintained their normal shape and the fluorescence was confined to the nucleus. Accordingly, the fusion protein constructs described herein are useful in methods for monitoring apoptosis in living cells.

### Example 4

### Construction of ptrEGFP-HMGB1 Expression Plasmid

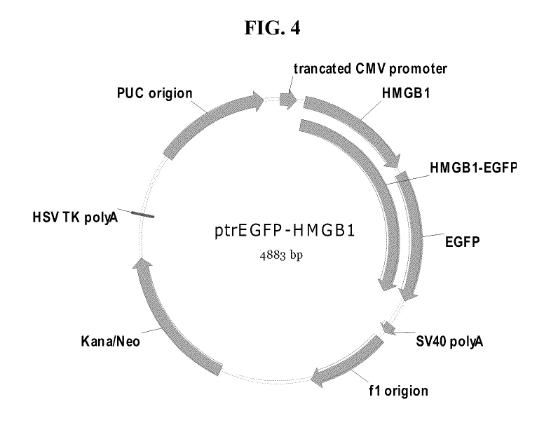

The HMGB1 cDNA sequence was assembled from genomic DNA as described in Example 1 and cloned into eukaryotic expression vector pEGFP-lac in which the HMGB1 gene was fused with EGFP and expressed by a truncated CMV early promoter and enhancer (FIG. 4, SEQ ID NO: 2).

### Example 5

### Imaging the Chromosomes of Living Cells

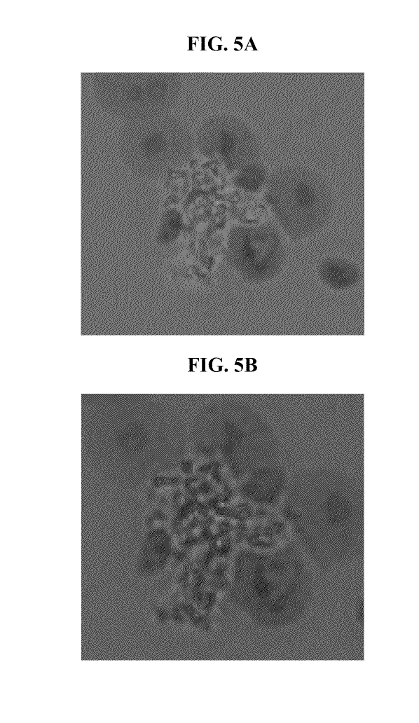

Cells (e.g., HEK 293FT) were transfected with the ptrEGFP-HMGB1 plasmid prepared as described in Example 4. After forty-eight hours, the chromosomes were observed using a standard karyotyping procedure with colchicine. Fluorescence microscopy was used to image the cells. The results are depicted in FIG. 5A and FIG. 5B and show that the cells transfected with ptrEGFP-HMGB1 provide for distinguishable imaging of the chromosomes.

### Example 6

### Imaging Cells Contacted With Biological Effector Molecules

In this example, a cultured cancer cell line (for example, a cancer cell line selected from those listed in Table 2) is transfected with the nucleic acid construct encoding the imaging agent prepared as described in Example 1.

TABLE 2
Human Cancer Cell Lines

| Cell Line | Cancer Type |
|---|---|
| CCRF-CEM, HL-60(TB), K-562, MOLT-4, RPMI-8226, | Leukemia |
| P388, P388/ADR | |
| A549, EKVX, HOP-62, HOP-92, NCI-H226, NCI-H23, | Non-Small |
| NCI-H322M, NCI-H460, NCI-H522, LXFL 529 | Cell Lung |
| COLO 205, HCC-2998, HCT-116, HCT-15, HT29, | Colon |
| KM12, SW-620, DLD-1, KM20L2SNB-78, XF 498 | |
| SF-268, SF-295, SF-539, SNB-19, SNB-75, U251 | CNS |
| LOX IMVI, MALME-3M, M14, SK-MEL-2, SK-MEL- | Melanoma |
| 28, SK-MEL-5, UACC-257, UACC-62, RPMI-7951, | |
| M19-MEL | |
| IGR-OV1, OVCAR-3, OVCAR-4, OVCAR-5, OVCAR-8, | Ovarian |
| SK-OV-3 | |
| 786-0, A498, ACHN, CAKI-1, RXF 393, SN12C, TK-10, | Renal |
| UO-31, RXF-631, SN12K1 | |
| PC-3, DU-145, MCF7 | Prostate |
| NCI/ADR-RES, MDA-MB-231, HS 578T, MDA-MB- | Breast |
| 435, MDA-N, BT-549, T-47D, MDA-MB-468 | |
| DMS 114, SHP-77 | Small Cell |
| | Lung |

The transfected cell is then split into two cultures. In one culture, the cells are contacted with an appropriate dosage of a biological effector molecule (e.g., a chemotherapeutic agent or other small molecule, or a DNA- or RNA-based pharmaceutical). The cells of the second culture are untreated. After a period of time (1 min, 30 min, 1 h, 1 d, 1 week, etc.), the cells are imaged using fluorescence microscopy. The integrity of the nucleus is observed, as well as the structure and dynamics of the chromosomes. Observations with regard to chromosome structure and dynamics in the treated cells are made and compared to the untreated cells. Data is quantified by measuring the pixels and/or intensity of fluorescent light in the image.

The efficacy of the biological effector molecule on inducing apoptosis or another nucleus-associated change in the cancer cell line is assessed by reduction in the amount or change in location (i.e., diffusion) of fluorescence in the cells of the treated culture compared to the untreated control culture.

### Equivalents

The present disclosure is not to be limited in terms of the particular embodiments described in this application, which are intended as illustrations of individual aspects thereof. Many modifications and variations can be made without departing from its spirit and scope, as will be apparent to those skilled in the art. Functionally equivalent embodiments within the scope of the disclosure, in addition to those enumerated herein, will be apparent to those skilled in the art from the foregoing descriptions. Such modifications and variations are intended to fall within the scope of the claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An imaging agent comprising a fusion protein having one or more DNA binding domains and one or more fluorescent domains, wherein the fusion protein is configured to localize in the nucleus of a cell.

2. The imaging agent of claim 1, wherein the DNA binding domain is selected from the group consisting of: an HMGB1; an HMGB2; and a histone H1.

3 The imaging agent of claim 1, wherein the fluorescent domain is selected from the group consisting of: a GFP; an EGFP; a YFP; an EYFP; a CFP; an ECFP; a BFP; an EBFP; an RFP; and a DsRed.

4. The imaging agent of claim 1, wherein the DNA binding domain is HMGB1 and the fluorescent domain is EGFP.

5. The imaging agent of claim 1 further comprising a nuclear localization domain.

6. The imaging agent of claim 5, wherein the nuclear localization domain is a nuclear localization sequence from the SV40 virus.

7. The imaging agent of claim 1 further comprising a linker peptide between the DNA binding domain and the fluorescent domain.

8. The imaging agent of claim 1 further comprising a cell-penetrating peptide.

9. The imaging agent of claim 8, wherein the cell-penetrating peptide is selected from the group consisting of: an antennapedia; a TAT; a transportan; and a polyarginine.

10. A cell comprising the imaging agent of claim 1.

11. A nucleic acid construct comprising a promoter operably linked to one or more DNA binding domains, one or more fluorescent domains and at least one nuclear localization domain.

12. The nucleic acid construct of claim 11, wherein the promoter is selected from the group consisting of: a strong constitutive promoter; a weak constitutive promoter; a tissue-specific promoter; and an inducible promoter.

13. The nucleic acid construct of claim 12, wherein the strong constitutive promoter is a CMV early promoter.

14. The nucleic acid construct of claim 12, wherein the weak constitutive promoter is a truncated CMV early promoter.

15. The nucleic acid construct of claim 11, wherein the DNA binding domain is HMGB1 and the fluorescent domain is EGFP.

16. A vector comprising the nucleic acid construct of claim 11.

17. The vector of claim 16, having the sequence according to SEQ ID NO: 1 or SEQ ID NO: 2.

18. A method for imaging a cell comprising:
(a) introducing into a cell:
(i) an imaging agent comprising a fusion protein having one or more DNA binding domains and one or more fluorescent domains, wherein the fusion protein is configured to localize in the nucleus of a cell; or
(ii) a nucleic acid construct for expression of an imaging agent comprising a promoter operably linked to one or more DNA binding domains, one or more fluorescent domains and at least one nuclear localization domain; and
(b) detecting the fluorescence of the imaging agent.

19. The method of claim 18, wherein the detecting comprises imaging the nucleus of a cell.

20. The method of claim 18, wherein the detecting comprises imaging the chromosomes of a cell.
